(19) United States
(12) Patent Application Publication
(10) Pub. No.: US 2018/0199925 A1
(43) Pub. Date: Jul. 19, 2018

(54) PLUG FOR AMNIOTIC RECOVERY

(71) Applicant: POSTECH ACADEMY-INDUSTRY FOUNDATION, Pohang-si (KR)

(72) Inventors: Hyeon Ji KIM, Pohang-si (KR); Hyun Sun KO, Pohang-si (KR); JinAh JANG, Pohang-si (KR); Jong Chui SHIN, Pohang-si (KR); Dong Woo CHO, Pohang-si (KR); Dong Heon HA, Pohang-si (KR)

(21) Appl. No.: 15/869,071

(22) Filed: Jan. 12, 2018

(30) Foreign Application Priority Data
Jan. 13, 2017 (KR) 10-2017-0006198

(51) Int. Cl. A61B 17/00; A61L 31/16; A61L 31/14; A61B 17/42

(52) U.S. Cl. CPC A61B 17/0057; A61L 31/16; A61L 31/148; A61B 2017/00893; A61M 2210/14; A61B 2017/00588; A61B 2017/00601; A61B 17/42

(57) ABSTRACT

Disclosed is a plug for amniotic recovery including a body part which blocks a wounded part of an amniotic membrane to recover the wounded part and a shock-absorbing part which is provided at one end of at least one of both ends in an insertion direction of the body part to absorb a shock by external force.

### CROSS-REFERENCE TO RELATED APPLICATION

This application claims under 35 U.S.C. § 119(a) the benefit of Korean Patent Application No. filed on, the entire contents of which are incorporated herein by reference.

### BACKGROUND

### (a) Technical Field

The present invention relates to a plug for amniotic recovery and more particularly, to a plug for amniotic recovery capable of preventing damage to an amniotic membrane by inserting the plug into a part of the amniotic membrane which is to be wounded or damaged in advance and providing the most similar environment to an original tissue and providing high recovery force to the amniotic membrane by releasing an amniotic membrane material inserted therein to the outside.

### (b) Background Art

In general, one of the greatest difficulties experienced in the medical treatment of obstetrics and gynecology is undesired pregnancy interruption and premature birth due to premature rupture of membranes (PROM).

The PROM is a symptom in which an amniotic membrane surrounding an amniotic cavity is ruptured before full term birth irrespective of gestational weeks and then an amniotic fluid is leaked, and occurs before 37 weeks of pregnancy without preterm labor and most frequently occurs before 28 weeks of pregnancy so that prognosis for a neonate is very poor. Recently, the rupture of the amniotic membrane is increasing during antenatal consultation, such as amniocentesis and umbilical cord paracentesis.

The PROM occupies about 3% of the entire pregnant women, but occupies 30% of premature births, 60% of perinatal deaths, and 50% of total neonatal neurological diseases.

Currently, treatment for the PROM is limited, and medical treatment is still difficult in addition to conservative treatment, antibiotics and steroid administration.

### SUMMARY OF THE DISCLOSURE

The present has been made in an effort to provide a plug for amniotic recovery capable of providing high recovery force to an amniotic membrane by inserting the plug into a part of the amniotic membrane which is to be wounded or damaged to release an amniotic membrane material inserted therein to the outside.

The objects of the present invention are not limited to the aforementioned effects, and other objects, which are not mentioned above, will be apparent to a person having ordinary skill in the art from the following description.

An exemplary embodiment of the present invention provides a plug for amniotic recovery including a body part which blocks a wounded part of an amniotic membrane to recover the wounded part and a shock-absorbing part which is provided at one end of at least one of both ends in an insertion direction of the body part to absorb a shock by external force.

An amniotic membrane therapeutic agent for treating the wounded part of the amniotic membrane may be provided at one side of the body part.

The body part may have at least one or more through holes, and an amniotic for treating the damaged amniotic membrane may be provided inside the body part.

The amniotic membrane therapeutic agent may be a gel or capsule type which is dissolved in the human body.

The shock-absorbing part may have a coil shape.

The plug for amniotic recovery of the present invention has the following effects.

First, the amniotic membrane therapeutic agent made of an antibiotic, an amniotic membrane therapeutic material, and the like is provided at one side of the plug for amniotic recovery, thereby rapidly treating and recovering the damaged amniotic membrane.

Second, the amniotic membrane therapeutic material is provided inside the body part in a gel or capsule type to be dissolved after inserted into the human body, thereby preventing the amniotic membrane therapeutic material from being lost when the plug for amniotic recovery is inserted.

Third, the plug for amniotic recovery is made of a material to be dissolved when staying for a predetermined time in a body, and thus, a separate removal surgery is not required.

Fourth, the shock-absorbing part for absorbing the shock is formed at both ends of the body part, thereby preventing a wound from occurring in the organs of the fetus and the pregnant woman or preventing the plug for amniotic recovery from being damaged even though the organs and the like of the fetus and the pregnant woman are in contact with the plug for amniotic recovery.

The effects of the present invention are not limited to the aforementioned effects, and other effects, which are not mentioned above, will be apparent to a person having ordinary skill in the art from the description of claims.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a perspective view of a plug for amniotic recovery according to the present invention;

FIG. 2 is a cross-sectional view of the plug for amniotic recovery according to the present invention; and

FIG. 3 is a use state diagram of the plug for amniotic recovery according to the present invention.

### DETAILED DESCRIPTION

Hereinafter, preferred exemplary embodiments of the present invention will be described in detail with reference to the accompanying drawings.

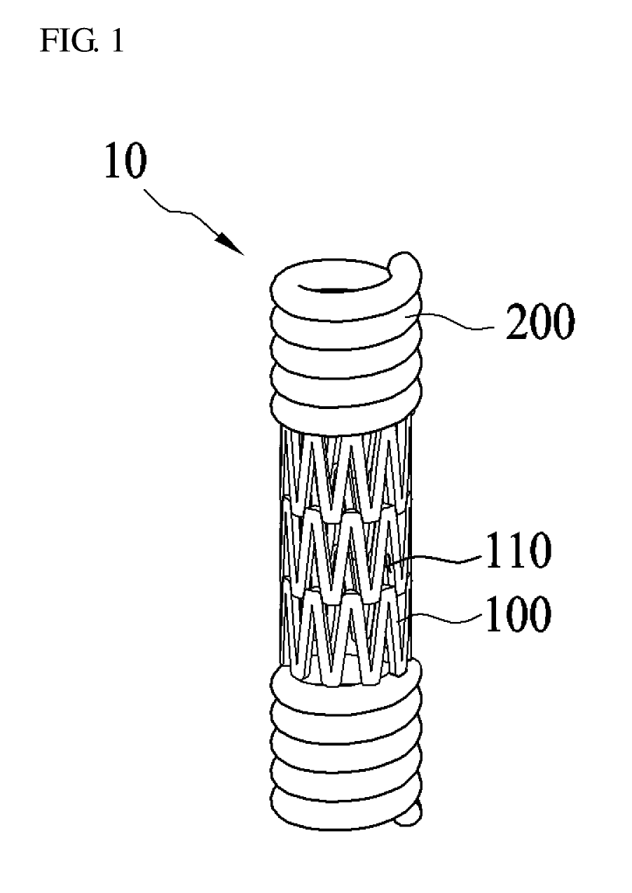

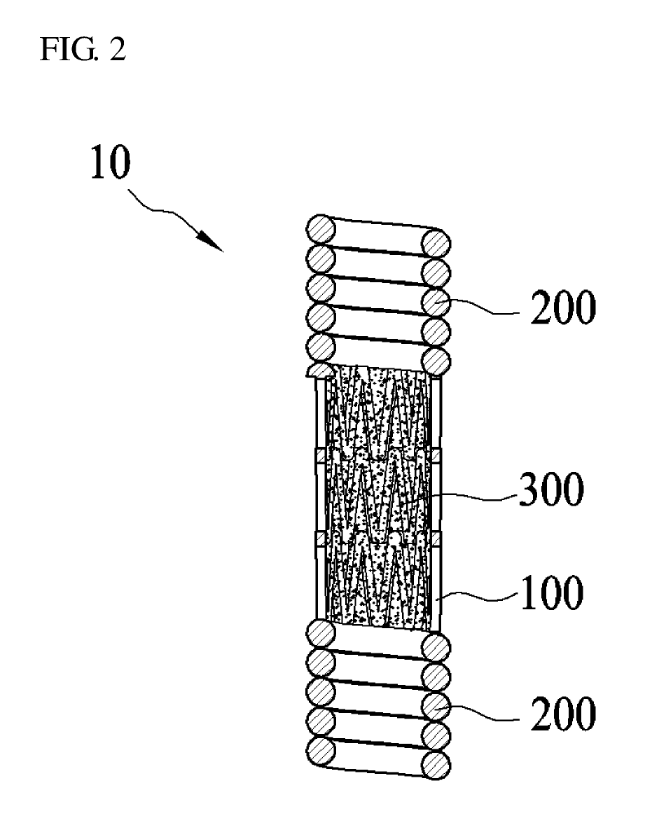

FIG. 1 is a perspective view of a plug for amniotic recovery according to the present invention and FIG. 2 is a cross-sectional view of the plug for amniotic recovery according to the present invention. A plug 10 for amniotic recovery according to the present invention is largely configured by a body part 100, a shock-absorbing part 200, and an amniotic membrane therapeutic agent 300, as shown in the FIGS. 1 and 2.

The body part 100 is a device which is inserted into a damaged part or a part to be damaged of an amniotic membrane A to prevent the amniotic membrane A from being damaged beforehand or recover the damaged amniotic membrane A. Such a body part 100 may have any shape as long as the body part 100 is inserted into a human body to prevent a damaged part of the amniotic membrane A. However, the body part 100 may have a tube or pipe shape having a predetermined length so as to be easily inserted into the human body through a tube and the like. In this case, a hollow part is formed in the body part 100 to be inserted with the amniotic membrane therapeutic agent 300 for treating and recovering the damaged part of the amniotic membrane A, and at least one or more through holes 110 may be formed in one side so that the amniotic membrane therapeutic agent 300 inserted therein may be released to the outside.

As one example, the body part 100 may have a shape such as a stent or a mash as illustrated in FIGS. 1 and 2, and may be formed to have a plurality of through holes 110 in a tube shape according to a use state. Such a body part 100 may be manufactured using any method, but may be manufactured using 3D printing as an example.

Further, the body part 100 may be made of any material, but preferably, may be made of a material which may be dissolved by a human's temperature after the amniotic membrane therapeutic agent 300 inserted into the human body is released. As an example, the body part 100 may be made of a polycaprolactone (PCL) material and the material of the body part 100 is not limited and any material may be used as long as the material is a harmless material of the human body. Further, according to the use state, the body part 100 may also be made of a material which is not dissolved in the body of a pregnant woman.

The shock-absorbing part 200 is provided in at least one end of both ends in an inserting direction of the body part 100 and absorbs external force due to collision so as to prevent a wound from being caused or the body part 100 from being damaged when organs and the like of the fetus or the pregnant woman are in contact with the body part 100.

Such a shock-absorbing part 200 may be formed of any material and shape as long as the shock-absorbing part 200 may absorb the external force generated by collision with the organs and the like of the fetus or the pregnant woman. As an example, the shock-absorbing part 200 is integrally formed with the body part 100 with the same material as the body part 100 and may be manufactured using 3D printing in a shape to absorb a shock generated at the outside. The shape of the shock-absorbing part 200 may be formed, for example, in a coil spring shape in which a plurality of coils is wound. It is apparent that the shock-absorbing part 200 may be separately formed from the body part 100 to be mutually coupled with the body part 100 according to the use state.

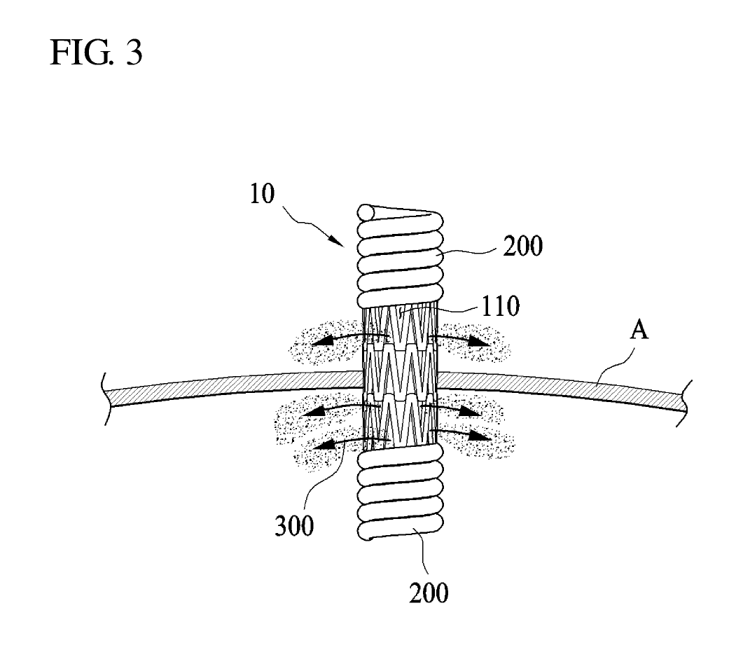

FIG. 3 is a use state diagram of the plug for amniotic recovery according to the present invention. The amniotic membrane therapeutic agent 300 is provided at one side of the body part 100 to treat the wound or the damaged part of the amniotic membrane A. The amniotic membrane therapeutic agent 300 may be made of various drugs such as an antibiotic and an amniotic membrane tissue regenerating material in consideration of the degree of damage of the amniotic membrane A, the constitution of a pregnant woman, or the like. In this case, the amniotic membrane therapeutic agent 300 may also be made of any material such as liquid and solid materials, but preferably, may be formed in a gel or capsule type which is dissolved in the human body and provided to be inserted into the hollow part in the body part 100. As such, the amniotic membrane therapeutic agent 300 formed in the gel or capsule type is dissolved by the temperature of the human body or body fluids and released to the outside of the body part 100 though the through holes 110 of the body part 100 to treat or regenerate the damaged part of the amniotic membrane A, as illustrated in FIG. 3.

The use state of the plug 10 for amniotic recovery configured by the aforementioned configuration is similar to an insertion method of a general stent and will be described in more detail as follows.

First, while the plug 10 for amniotic recovery is mounted on a body insertion device, the plug 10 for amniotic recovery moves to the damaged part of the amniotic membrane A through a part of the body of the pregnant woman.

Thereafter, the plug 10 for amniotic recovery is inserted and fixed by passing through the damaged part of the amniotic membrane A by a method of pushing the plug 10 for amniotic recovery through the body insertion device.

Finally, the body insertion device is removed to complete the insertion of the plug 10 for amniotic recovery.

The amniotic membrane therapeutic agent 300 provided in the body part 100 of the plug 10 for amniotic recovery inserted through the aforementioned process is dissolved by the body temperature or body fluids to be released to the outside of the body part 100 through the through holes 110 of the body part 100. As such, the released amniotic membrane therapeutic agent 300 treats and recovers the damaged part of the amniotic membrane A.

Even though the organs and the like of the fetus or the pregnant woman are in contact with the plug 10 for amniotic recovery by movement such as the behavior of the pregnant woman after the plug 10 for amniotic recovery is inserted, the shock-absorbing part 200 formed in the body part 100 absorbs the shock, thereby preventing occurrence of a wound in the fetus or the pregnant woman and preventing the damage to the plug 10 for amniotic recovery.

If the materials of the body part 100 and the shock-absorbing part 200 of the plug 10 for amniotic recovery are made of a material which is dissolvable in the human body, the materials may be dissolved simultaneously with treatment and recovery of the amniotic membrane A of the amniotic membrane therapeutic agent 300.

As described above, those skilled in the art to which the present invention belongs will be able to understand that the present invention can be implemented in other detailed forms without changing the technical spirit or an essential characteristic. Therefore, it should be understood that the aforementioned exemplary embodiments are just illustrative and not restrictive in all aspects. The scope of the present invention is defined by not the specification, but the following claims, and all of changes and modifications obtained from the meaning and range of claims and equivalent concepts should be construed as being included in the scope of the present invention.

What is claimed is:

1. A plug for amniotic recovery, comprising:
a body part which blocks a wounded part of an amniotic membrane to recover the wounded part; and
a shock-absorbing part which is provided at one end of at least one of both ends in an insertion direction of the body part to absorb a shock by external force.

2. The plug for amniotic recovery of claim 1, wherein an amniotic membrane therapeutic agent for treating the wounded part of the amniotic membrane is provided at one side of the body part.

3. The plug for amniotic recovery of claim 1, wherein the body part has at least one or more through holes, and an amniotic membrane therapeutic agent for treating the wounded part of the amniotic membrane is provided inside the body part.

4. The plug for amniotic recovery of claim 3, wherein the amniotic membrane therapeutic agent is a gel or capsule type which is dissolved in the human body.

5. The plug for amniotic recovery of claim 1, wherein the shock-absorbing part has a coil shape.

6. The plug for amniotic recovery of claim 1, wherein the body part and the shock-absorbing part are made of materials dissolvable in the body.
